(19) United States
(12) Patent Application Publication
(10) Pub. No.: US 2014/0081159 A1
(43) Pub. Date: Mar. 20, 2014

(54) NON-INVASIVE CONTINUOUS BLOOD PRESSURE MONITORING SYSTEM AND METHOD

(71) Applicant: HOLUX TECHNOLOGY INC., Hsinchu (TW)

(72) Inventors: TEH HO TAO, MIAOLI COUNTY (TW); SHIH JEN HU, TAINAN CITY (TW); NENG YU PAN, NEW TAIPEI CITY (TW)

(73) Assignee: HOLUX TECHNOLOGY INC., HSINCHU (TW)

(21) Appl. No.: 13/796,899

(22) Filed: Mar. 12, 2013

Related U.S. Application Data
(60) Provisional application No. 61/701,981, filed on Sep. 17, 2012.

(51) Int. Cl. A61B 5/021

(52) U.S. Cl. CPC A61B 5/02125
USPC 600/493

(57) ABSTRACT

A non-invasive continuous blood pressure monitoring system includes a first sensor configured to send a first ultra-wideband electromagnetic pulses to an upper site of a blood vessel and receive a first reflected ultra-wideband electromagnetic pulses with phase variations caused by a pressure wave propagating in the blood vessel, a second sensor configured to send a second ultra-wideband electromagnetic pulses to a lower site of the blood vessel and receive a second reflected ultra-wideband electromagnetic pulses with phase variations caused by the pressure wave propagating in the blood vessel, and a signal-processing and blood pressure displaying device configured to calculate an estimated blood pressure by taking into consideration a propagation time of the pressure wave from the upper site to the lower site in the blood vessel.

### BACKGROUND

1. Technical Field

The present invention relates to a non-invasive continuous blood pressure monitoring system and method, and more particularly, to a non-invasive continuous blood pressure monitoring system and method of implementing the blood pressure measurement by taking into consideration a propagation time of the pressure wave from an upper site to a lower site in the same blood vessel.

2. Description of Related Arts

Monitoring blood pressure of systematic arteries, including the aorta and branch arteries, plays an essential role in clinical medicine. Traditional monitoring methods include (1) inserting an invasive cannula into a patient's radial artery, transmitting blood pressure to an in vitro sensor, and converting the sensor output into a blood pressure value; (2) fastening a cuff over a brachial artery, applying external pressure to the artery to obtain a blood vessel pulse signal and converting the same into a blood pressure value. Disadvantages of the two monitoring methods are that (1) although the invasive method allows continuous monitoring, it requires a medical staff to perform operations and care for cleanness of the patent's wound to prevent infections; (2) the cuff method is not suitable for long-term continuous monitoring of blood pressure because constant long-term squeezing of a patient's arm may cause the patient to feel soreness and numbness in his/her arm.

In accordance with clinical studies, it is highly required that blood pressure variations of heart disease patients in a hospital or under home care to be monitored, and the most appropriate monitoring type is long-term continuous monitoring. From long term variations of blood pressure, primary hypertension can be recognized and by performing long-term monitoring of high blood pressure during a patient's sleep, strokes and kidney failures can be prevented. The aorta is the source for supplying blood to organ tissues of a human body, and therefore an accurate measurement of the aortic blood pressure serves as an important basis for a doctor's diagnosis and treatment of a patient's cardiovascular disease.

The conventional non-invasive method requires a cuff to be fastened on a particular part of a body, and each group of measurement data is obtained after going through periods of inflation and deflation. Therefore, it is impossible to continuously monitor a patient's blood pressure without interruption. If such a method is applied for long-term monitoring, the patient may feel extremely uncomfortable due to long-term squeezing of the patient's arm in cycles.

A method disclosed in U.S. Pat. Nos. 6,893,401 and 6,599,251 measures pulse wave signals from any two positions of a body to serve as basis for measuring propagation times for pressure waves. Since these two positions are not located at two ends of the same blood vessel, but rather at two branches of the systemic arteries, such as arteries at an earlobe and at a finger, the theoretical relationship between the propagation time and the blood pressure cannot be established, thus causing the accuracy of the blood pressure measured using this method to be relatively low.

### SUMMARY

One aspect of the present disclosure provides a non-invasive continuous blood pressure monitoring system and method of implementing the blood pressure measurement by taking into consideration a propagation time of the pressure wave from an upper site to a lower site in the same blood vessel.

A non-invasive continuous blood pressure monitoring system according to this aspect of the present disclosure comprises a first sensor configured to send a first ultra-wideband (UWB) electromagnetic pulses to an upper site of a blood vessel and receive from the same site a first reflected UWB electromagnetic pulses by a pressure wave propagating in the blood vessel; a second sensor configured to send a second UWB electromagnetic pulses to a lower site of the blood vessel and receive from the same site a second reflected UWB electromagnetic pulses by the pressure wave propagating in the blood vessel; and a signal-processing and blood pressure displaying device configured to calculate an estimated blood pressure by taking into consideration a propagation time of the pressure wave from the upper site to the lower site in the blood vessel.

A non-invasive continuous blood pressure monitoring method according to this aspect of the present disclosure comprises sending a first UWB electromagnetic pulses to an upper site of a blood vessel and receiving from the same site a first reflected UWB electromagnetic pulses by a pressure wave propagating in the blood vessel; sending a second UWB electromagnetic pulses to a lower site of the blood vessel and receiving from the same site a second reflected UWB electromagnetic pulses by the pressure wave propagating in the blood vessel; and calculating an estimated blood pressure by taking into consideration a propagation time of the pressure wave from the upper site to the lower site in the blood vessel.

According to one embodiment of the present invention, the estimated blood pressure can be obtained by performing a simple linear calculation from the propagation time of the pressure wave obtained from the upper site to the lower site in the same blood vessel. In contrast, the conventional measuring method disclosed in U.S. Pat. Nos. 6,893,401 and 6,599,251 needs to perform a very complicated non-linear calculation with a natural logarithm operation.

Because of the sensing characteristics of the UWB electromagnetic monitoring method, the present invention can also be applied to other sites such as the aorta or a leg artery. In contrast, the conventional measuring method disclosed in U.S. Pat. Nos. 6,893,401 and 6,599,251 can be applied to an earlobe, finger, and toe, but cannot be applied to the aorta or a leg artery. In addition, because of the sensing characteristics of the UWB electromagnetic monitoring method, the present invention can also be used to reduce discomfort caused by long-term application of the contact-type measuring method.

The foregoing has outlined rather broadly the features and technical advantages of the present disclosure in order that the detailed description of the disclosure that follows may be better understood. Additional features and advantages of the disclosure will be described hereinafter, which form the subject of the claims of the disclosure. It should be appreciated by those skilled in the art that the conception and specific embodiment disclosed may be readily utilized as a basis for modifying or designing other structures or processes for carrying out the same purposes of the present disclosure. It should also be realized by those skilled in the art that such equivalent constructions do not depart from the spirit and scope of the disclosure as set forth in the appended claims.

### BRIEF DESCRIPTION OF THE DRAWINGS

A more complete understanding of the present disclosure may be derived by referring to the detailed description and claims when considered in connection with the Figures, where like reference numbers refer to similar elements throughout the Figures, and:

FIG. 1 and FIG. 2 are schematic diagrams showing the application of a non-invasive continuous blood pressure monitoring system to a blood vessel according to one embodiment of the present invention;

FIG. 3 and FIG. 4 are functional block diagrams of the first sensor according to one embodiment of the present invention, and the second sensor may have the same design;

FIG. 5 is a disassembled diagram of the first sensor according to one embodiment of the present invention;

FIG. 6 shows the waveforms of the pressure wave signals measured by the first sensor and the second sensor according to one embodiment of the present invention;

FIG. 7 compares the measured pressure (transverse axis) from a cuff-type blood pressure monitor and the propagation times of a pressure wave (PWTT, vertical axis) from the non-invasive continuous blood pressure monitoring system 20 according to one embodiment of the present invention;

FIG. 8 compares the measured pressure (transverse axis) from a cuff-type blood pressure monitor and the calculated blood pressure from the propagation times of a pressure wave (PWTT, vertical axis) of the non-invasive continuous blood pressure monitoring system 20 according to one embodiment of the present invention; and

FIG. 9 and FIG. 10 are schematic diagrams showing the application of a non-invasive continuous blood pressure monitoring system to other sites such as the aorta or a leg artery according to one embodiment of the present invention.

### DETAILED DESCRIPTION

The following description of the disclosure accompanies drawings, which are incorporated in and constitute a part of this specification, and illustrate embodiments of the disclosure, but the disclosure is not limited to the embodiments. In addition, the following embodiments can be properly integrated to complete another embodiment.

References to “one embodiment,” “an embodiment,” “exemplary embodiment,” “other embodiments,” “another embodiment,” etc. indicate that the embodiment(s) of the disclosure so described may include a particular feature, structure, or characteristic, but not every embodiment necessarily includes the particular feature, structure, or characteristic. Further, repeated use of the phrase “in the embodiment” does not necessarily refer to the same embodiment, although it may.

The present disclosure is directed to a non-invasive continuous blood pressure monitoring system and method. In order to make the present disclosure completely comprehensible, detailed steps and structures are provided in the following description. Obviously, implementation of the present disclosure does not limit special details known by persons skilled in the art. In addition, known structures and steps are not described in detail, so as not to limit the present disclosure unnecessarily. Preferred embodiments of the present disclosure will be described below in detail. However, in addition to the detailed description, the present disclosure may also be widely implemented in other embodiments. The scope of the present disclosure is not limited to the detailed description, and is defined by the claims.

Based on theory, it can be derived that a propagation time of a pressure wave in an artery is inversely proportional to blood pressure. However, because the current measuring technique for a propagation time of a pressure wave in a blood vessel is insufficiently accurate, significant errors may occur. The reason is that such methods are limited to using an R wave of an electrocardiogram (ECG) as a basis, and therefore the measured values are not the actual propagation time of the arterial pressure wave. Ultra-wideband (UWB) electromagnetic pulses can be used to capture instant pressure wave signals from contractions at two ends of a blood vessel, thereby solving the foregoing measuring error.

The present invention is directed to a non-invasive continuous blood pressure monitoring system and method that adopt a pulse detection method using two sensors to synchronously capture propagating times of pressure waves from two ends of the systematic arteries of a human body so that a blood pressure value can be calculated directly without the need for applying external pressure.

The present invention proposes to use a UWB electromagnetic measuring technique to implement a continuous blood pressure monitor. The principle of this measuring technique involves sending short UWB electromagnetic pulses (preferably with a pulse width shorter than 2 nanoseconds) over a transmitting antenna to a blood vessel, and receiving a reflective UWB electromagnetic pulses from the blood vessel over the receiving antenna. The vibration of the blood vessel causes a phase variation Δφ in the reflective UWB electromagnetic pulses, and the small phase variation is directly proportional to a vibration displacement X(t) of the blood vessel. Consequently, a pressure wave signal of a blood vessel can be obtained from a phase recovery processing method, which is disclosed in EP patent applications (EP1803396A1 and EP2093588A1) of the present inventor, and both EP patent applications are herein incorporated by reference in its entirety.

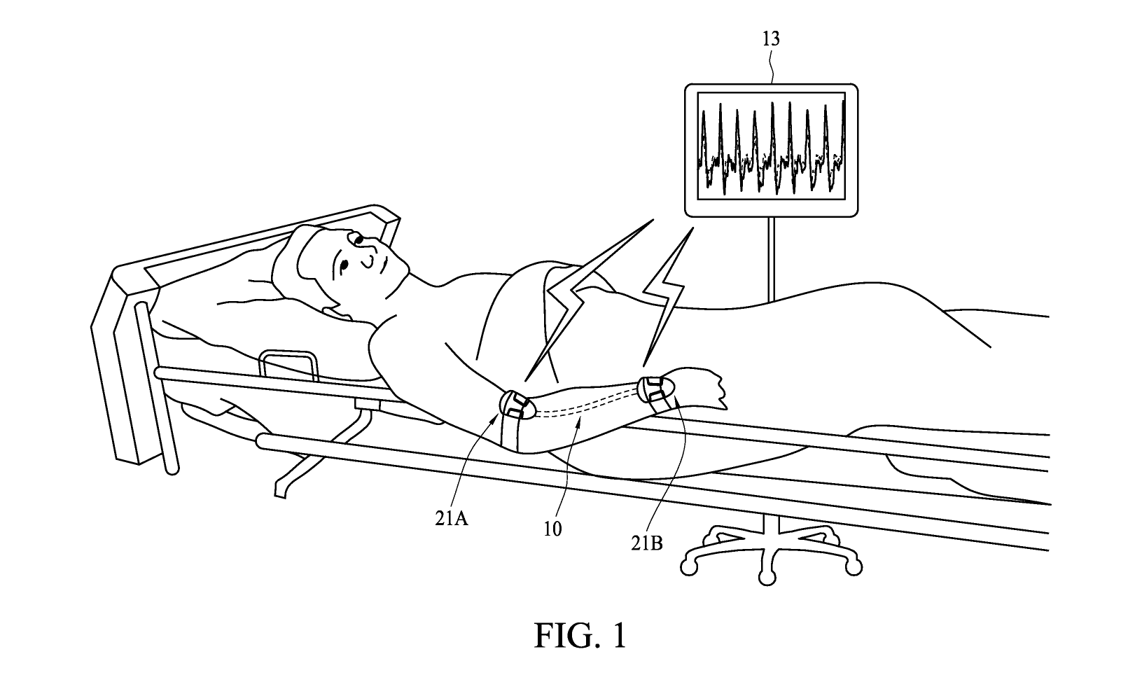

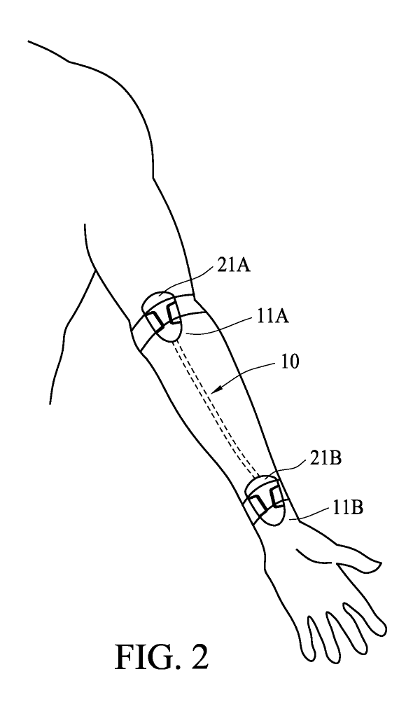

FIG. 1 and FIG. 2 are schematic diagrams showing the application of a non-invasive continuous blood pressure monitoring system 20 to a blood vessel 10 according to one embodiment of the present invention. In an exemplary embodiment of the present invention, the non-invasive continuous blood pressure monitoring system 20 comprises a first sensor 21A configured to send a first UWB electromagnetic pulses to an upper site 11A of a blood vessel 10 and receive from upper site 11A a first reflected UWB electromagnetic pulses with phase variations caused by a pressure wave propagating in the blood vessel 10; a second sensor 21B configured to send a second UWB electromagnetic pulses to a lower site 11B of the blood vessel 10 and receive from lower site 11B a second reflected UWB electromagnetic pulses with phase variations caused by the pressure wave propagating in the blood vessel 10; and a signal-processing and blood pressure displaying device 13 configured to calculate an estimated blood pressure by taking into consideration a propagation time of the pressure wave from the upper site 11A to the lower site 11B in the blood vessel 10.

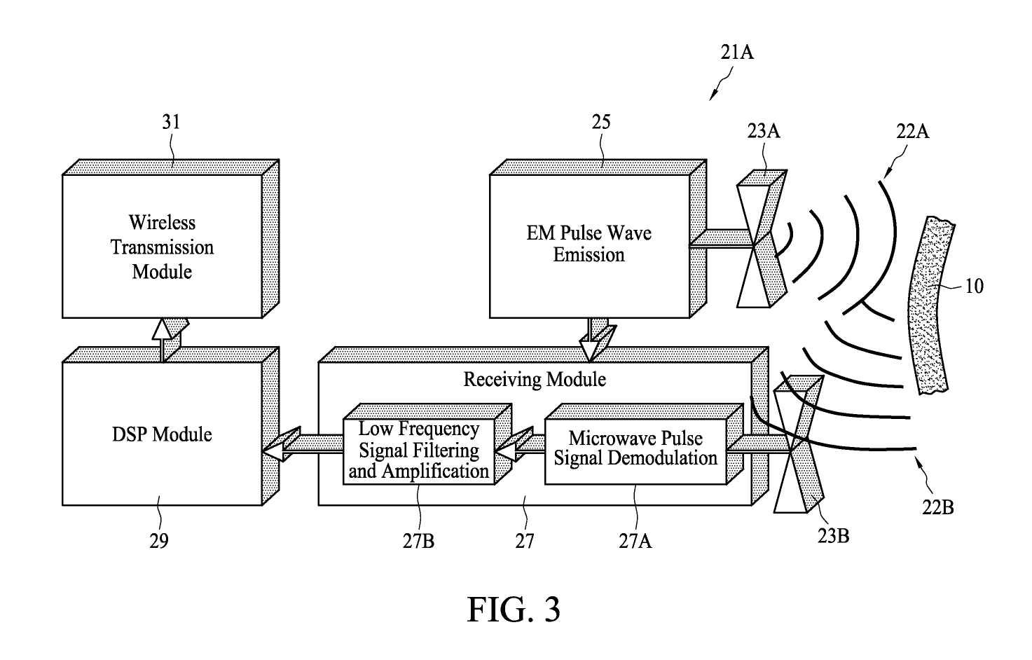

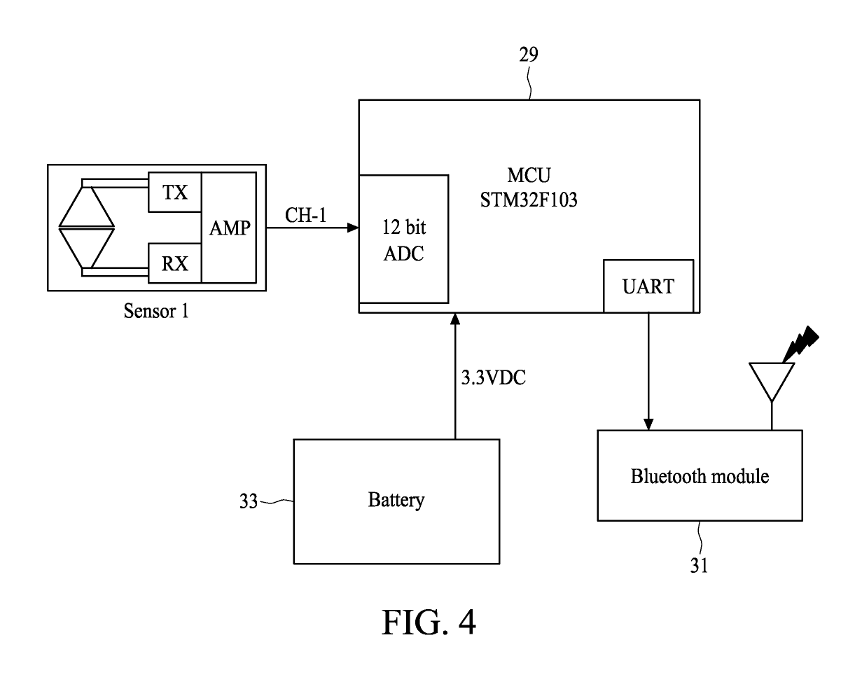

FIG. 3 and FIG. 4 are functional block diagrams of the first sensor 21A according to one embodiment of the present invention, and the second sensor 21B may have the same design. In an exemplary embodiment of the present invention, the first sensor 21A comprises a transmitting antenna 23A configured to send the UWB electromagnetic pulses 22A from a pulse source 25 to the blood vessel 10, a receiving antenna 23B configured to receive the reflected UWB electromagnetic pulses with phase variations caused by the pressure wave propagating in the blood vessel 10, a receiving module 27 connected to the receiving antenna 23B, a digital signal processing module 29 connected to the receiving module 27, and a wireless transmission module 31 connected to the digital signal processing module 29. In one embodiment of the present invention, the wireless transmission module 31 can be a Bluetooth module, and the receiving module 27 may comprise a signal demodulation unit 27A and a filtering and amplification unit 27B.

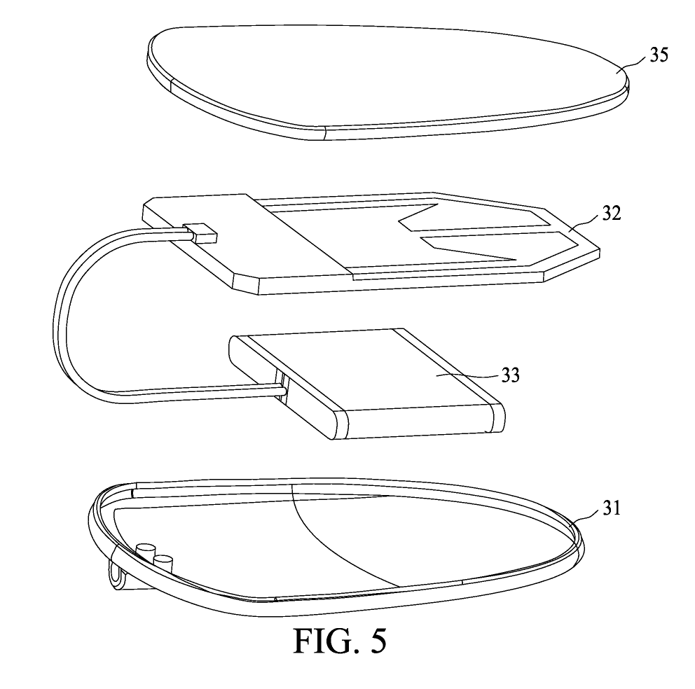

FIG. 5 is a disassembled diagram of the first sensor 21A according to one embodiment of the present invention, and the second sensor 21B may have the same design. In an exemplary embodiment of the present invention, the first sensor 21A comprises a bottom enclosure 31, a circuit board 32 with the electronics thereon, a lithium (Li) ion battery 33 and a top enclosure 35.

Referring back to FIG. 1 and FIG. 2, the first sensor 21A is placed at an elbow to capture the pressure wave signal at an elbow end of a radial artery of a forearm, and the second sensor 21B is arranged at a wrist to detect the same pressure wave signal at a wrist end of the radial artery of the forearm. In an exemplary embodiment of the present invention, output signals of the first sensor 21A and the second sensor 21B are wirelessly transmitted to the signal-processing device 13 through the wireless transmission module 31, respectively, and synchronization of the two sets of signals is performed in the signal-processing and blood pressure displaying device 13.

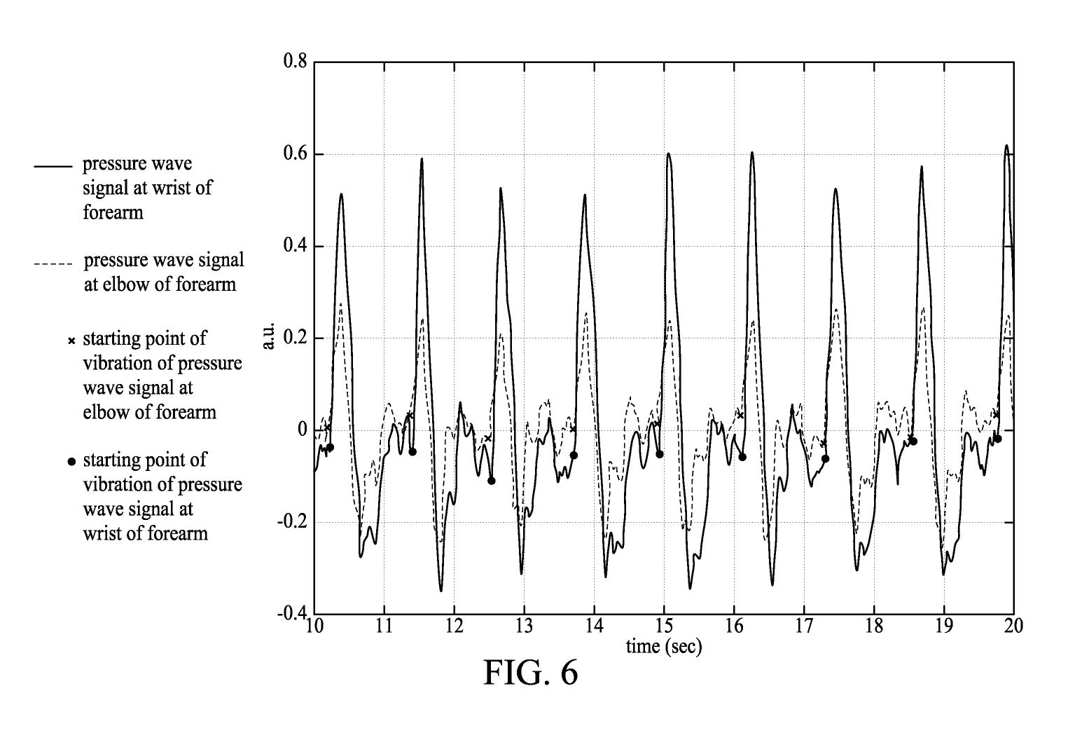

FIG. 6 shows the waveforms of the pressure wave signals measured by the first sensor 21A and the second sensor 21B according to one embodiment of the present invention. In an exemplary embodiment of the present invention, the embedded blood pressure monitoring software of the signal-processing and blood pressure displaying device 13 applies a moving average filtering technique to the received signals of the two sensors wirelessly transferred from wireless transmission module 31. As shown in FIG. 6, the time interval between the starting points of the two sensor signals is calculated as the propagation time of the blood pressure wave. Subsequently, the blood pressure value (P) is calculated by using a linear formula.

P=Po−β×PWTT

wherein Po and β are constant, and PWTT represents the propagation time of the pressure wave from the upper site 11A to the lower site 11B in the blood vessel 10.

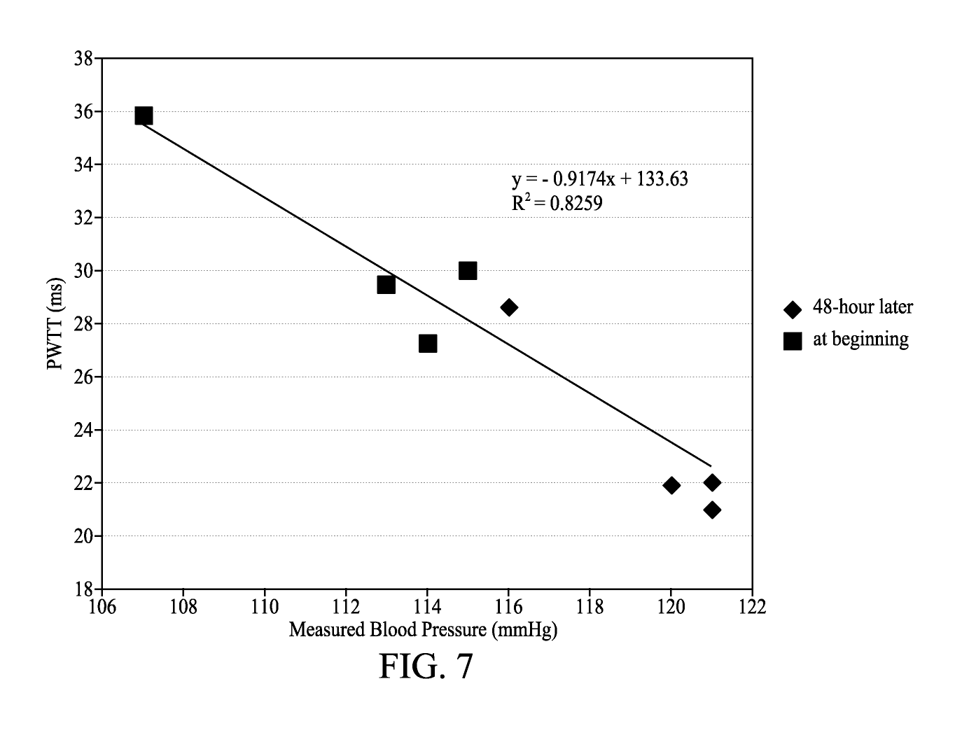

FIG. 7 compares the measured pressure (transverse axis) from a cuff-type blood pressure monitor and the propagation times of a pressure wave (PWTT, vertical axis) from the non-invasive continuous blood pressure monitoring system 20 according to one embodiment of the present invention. In order to verify the stability of the measurement for propagation times of pressure waves, a cuff-type blood pressure monitor and the measurement for propagation times of pressure waves are concurrently applied to monitor a subject. Two test results separated by an interval of 48 hours are shown in FIG. 7, wherein the testing result at the beginning is represented by the square, and the testing result for 48-hours later is represented by the diamond. As shown in FIG. 7, under a long-term condition, the parameters Po and β for converting the propagation times of the pressure waves into blood pressure can be maintained invariably.

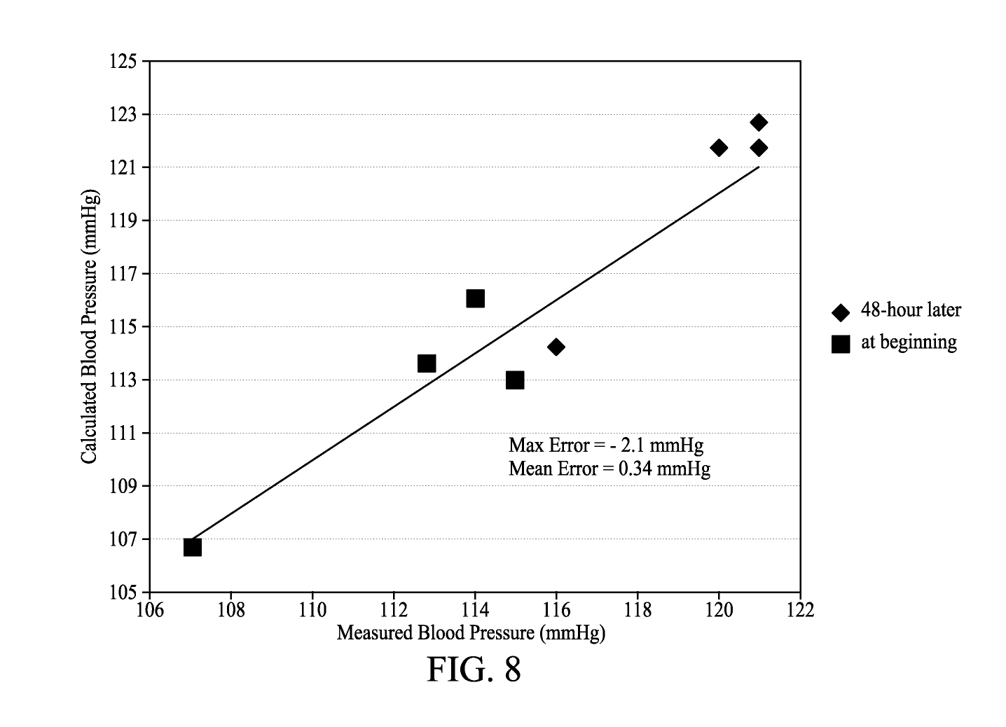

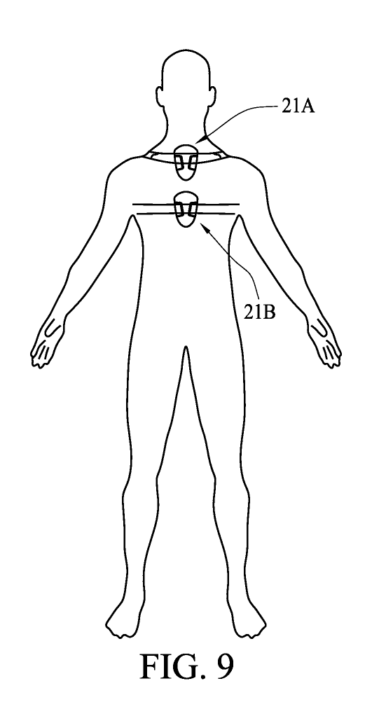

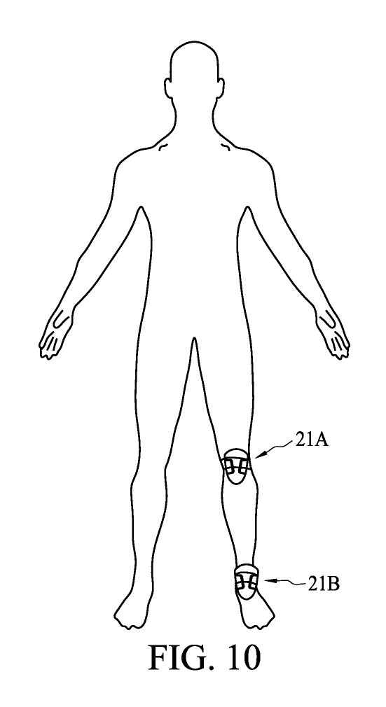

FIG. 8 compares the measured pressure (transverse axis) from a cuff-type blood pressure monitor and the calculated blood pressure from the propagation times of pressure wave (PWTT, vertical axis) of the non-invasive continuous blood pressure monitoring system 20 according to one embodiment of the present invention. As shown in FIG. 8, a variation range of the systolic pressure measurements of the same subject separated by an interval of 48 hours is 14 mmHg, and a high correlation coefficient R of 0.91 between the measured values of propagation times of the pressure waves and the cuff-type blood pressure monitor can be maintained, with the mean error being 0.34 mmHg, which is within the error range of ±5 mmHg as prescribed by the ANSI SP-10 standard. Therefore, a long-term accurate blood pressure monitor can be implemented with a single set of parameters Po and β. The above-mentioned method can also be applied to other sites such as the aorta or a leg artery to perform accurate blood pressure monitoring, as shown in FIG. 9 and FIG. 10.

According to one embodiment of the present invention, the estimated blood pressure can be obtained by performing a simple linear calculation from the propagation time of the pressure wave (PWTT) obtained from the upper site 11A to the lower site 11B in the same blood vessel 10. In contrast, the conventional measuring method disclosed in U.S. Pat. Nos. 6,893,401 and 6,599,251 needs to perform a very complicated non-linear calculation with a natural logarithm operation.

The present invention can also be applied to other sites such as the aorta or a leg artery. In contrast, the conventional measuring method disclosed in U.S. Pat. Nos. 6,893,401 and 6,599,251 can be applied to an earlobe, finger, and toe, but cannot be applied to the aorta or a leg artery. In addition, because of the sensing characteristic of the UWB RF monitoring method, the present invention can also be used to reduce discomfort caused by long-term application of the contact-type measuring method.

Although the present disclosure and its advantages have been described in detail, it should be understood that various changes, substitutions and alterations can be made herein without departing from the spirit and scope of the disclosure as defined by the appended claims. For example, many of the processes discussed above can be implemented in different methodologies and replaced by other processes, or a combination thereof.

Moreover, the scope of the present application is not intended to be limited to the particular embodiments of the process, machine, manufacture, composition of matter, means, methods and steps described in the specification. As one of ordinary skill in the art will readily appreciate from the disclosure of the present disclosure, processes, machines, manufacture, compositions of matter, means, methods, or steps, presently existing or later to be developed, that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A non-invasive continuous blood pressure monitoring system, comprising:
a first sensor configured to send a first UWB electromagnetic pulses to an upper site of a blood vessel and receive a first reflected UWB electromagnetic pulses with phase variations caused by a pressure wave propagating in the blood vessel;
a second sensor configured to send a second UWB electromagnetic pulses to a lower site of the blood vessel and receive a second reflected UWB electromagnetic pulses with phase variations caused by the pressure wave propagating in the blood vessel; and
a signal-processing and blood pressure displaying device configured to calculate an estimated blood pressure by taking into consideration a propagation time of the pressure wave from the upper site to the lower site in the blood vessel.

2. The non-invasive continuous blood pressure monitoring system of claim 1, wherein the signal-processing and blood pressure displaying device is configured to calculate the estimated blood pressure by performing a linear calculation.

3. The non-invasive continuous blood pressure monitoring system of claim 1, wherein the signal-processing and blood pressure displaying device is configured to calculate the estimated blood pressure by performing a calculation:
P=Po−β×PWTT
wherein Po and β are constant, and PWTT represents the propagation time of the pressure wave from the upper site to the lower site in the blood vessel.

4. The non-invasive continuous blood pressure monitoring system of claim 1, wherein the first sensor and the second sensor are positioned on a forearm artery.

5. The non-invasive continuous blood pressure monitoring system of claim 1, wherein the first sensor and the second sensor are positioned on a leg artery.

6. The non-invasive continuous blood pressure monitoring system of claim 1, wherein the first sensor and the second sensor are positioned on the aorta.

7. The non-invasive continuous blood pressure monitoring system of claim 1, wherein the first UWB electromagnetic pulses and the second UWB electromagnetic pulses have a pulse width shorter than 2 nanoseconds.

8. The non-invasive continuous blood pressure monitoring system of claim 1, further comprising a wireless transmission module configured to transmit the first reflected UWB electromagnetic pulses and the second reflected UWB electromagnetic pulses to the signal-processing and blood pressure displaying device.

9. A non-invasive continuous blood pressure monitoring method, comprising steps of:
sending a first UWB electromagnetic pulses to an upper site of a blood vessel and receiving a first reflected UWB electromagnetic pulses with phase variations caused by a pressure wave propagating in the blood vessel;
sending a second UWB electromagnetic pulses to a lower site of the blood vessel and receiving a second reflected UWB electromagnetic pulses with phase variations caused by the pressure wave propagating in the blood vessel; and
calculating an estimated blood pressure by taking into consideration a propagation time of the pressure wave from the upper site to the lower site in the blood vessel.

10. The non-invasive continuous blood pressure monitoring method of claim 9, wherein the calculating of the estimated blood pressure is performed by a linear calculation.

11. The non-invasive continuous blood pressure monitoring method of claim 9, wherein the calculating of the estimated blood pressure is performed by a calculation:
P=Po−β×PWTT
wherein Po and β are constant, and PWTT represents the propagation time of the pressure wave from the upper site to the lower site in the blood vessel.

12. The non-invasive continuous blood pressure monitoring method of claim 9, wherein the first UWB electromagnetic pulses and the second UWB electromagnetic pulses are sent to a forearm artery.

13. The non-invasive continuous blood pressure monitoring method of claim 9, wherein the first UWB electromagnetic pulses and the second UWB electromagnetic pulses are sent to a leg artery.

14. The non-invasive continuous blood pressure monitoring method of claim 9, wherein the first UWB electromagnetic pulses and the second UWB electromagnetic pulses are sent to the aorta.

15. The non-invasive continuous blood pressure monitoring method of claim 9, wherein the first UWB electromagnetic pulses and the second UWB electromagnetic pulses have a pulse width shorter than 2 nanoseconds.
